(19) United States
(12) Patent Application Publication
(10) Pub. No.: US 2012/0052141 A1
(43) Pub. Date: Mar. 1, 2012

(54) NOVEL USE OF COMPOUNDS FOR TREATING AND ALLEVIATING CELLULITE

(75) Inventors: Regina GORALCZYK, Grenzach-Wyhlen (DE); Joseph Schwager, Basel (CH); Karin Wertz, Rheinfelden (DE)

(73) Assignee: DSM IP ASSETS B.V., Heerleen (NL)

(21) Appl. No.: 13/289,128

(22) Filed: Nov. 4, 2011

Related U.S. Application Data
(62) Division of application No. 12/295,408, filed on Aug. 24, 2009, now abandoned.

(30) Foreign Application Priority Data
Mar. 31, 2006 (EP) 06006860.8

(51) Int. Cl. A61K 8/97; A61K 31/353; A61K 8/49; A61K 31/05; A61K 8/34; A61K 31/202; A61K 8/36; A61K 31/01; A61K 8/31; A61K 31/045; A61K 31/047; A61K 31/352; A61Q 19/06; A61P 17/00; C07D 311/62; C07C 39/21; C07C 39/11; C07C 35/21; C07C 11/21; A61K 36/738

(52) U.S. Cl. 424/765; 514/456; 514/733; 514/560; 514/731; 514/762; 514/729; 549/399; 568/729; 568/766; 568/816; 585/16

(57) ABSTRACT

The present invention is directed inter alia to the use of at least one compound selected from the group consisting of (−)-epigallocatechin gallate, resveratrol, eicosapentaenoic acid, docosahexaenoic acid, rose hip extract/concentrate, hydroxytyrosol, lycopene, lutein, β-cryptoxanthin, zeaxanthin and derivatives thereof, for the treatment of cellulite, for the prevention of the development of mild cellulite, for the prevention of the progression of mild cellulite to severe cellulite, for smoothening the micro relief of the skin, for maintaining or increasing the tensile properties of the skin, and/or for reducing the fat mass and the circumference at the hips and thighs, as well as to the corresponding cosmetic and medical methods.

This application is a divisional of application Ser. No. 12/295,408 filed Aug. 24, 2009, which in turn is the U.S. national phase of International Application No. PCT/EP2007/002950, filed 2 Apr. 2007, which designated the U.S. and claims priority to European Application No. 06006860.8 filed 31 Mar. 2006, the entire contents of each of which are hereby incorporated by reference.

Cellulite (gynoid lipodystrophy) is an alteration of the topography of the skin that occurs mainly in women in the pelvic region, lower limbs. It affects ˜85% of post-adolescent women. Cellulite is considered as a localized metabolic disorder of the subcutaneous tissue.

Typical features are structural alterations in the dermis, in the microcirculation and within the adipocyte, such as deterioration of the dermal matrix and vasculature, particularly loss of capillary networks, leading to excess fluid retention within dermal and subcutaneous tissue. This loss of the capillary network is thought to be due to engorged fat cells clumping together and inhibiting venous return.

The functional units of the fatty tissue which are involved in cellulite are the matricial-interstitial unit, the microcirculatory unit, the neuro-vegetative unit, hormonal factors and, predisposing genetic factors. Four pathophysiological evolutionary stages can be defined: pre-capillary arteriolar sphincter alterations lead to modification of the capillo-venular permeability, as well as to capillary ectasia with pericapillary and interadipocyte transudation and oedema. The oedema causes metabolic changes which result in hyperplasia and hypertrophy of the reticular framework. This leads to the formation of an irregular framework of pericapillary and pre-adipocyte argentaffin fibrils. Aniso-poikilocytotic adipocytes surrounded by reticular septae of irregular thickness are formed. Collagen fibres bind together around groups of adipocytes, forming micronodules. Sclerosis causes macronodules to form through the confluence of several micronodules. The structural alterations in the dermis cause the clinically visible mattress or orange peel like appearance of the skin.

By cellulite grading according to Nürnberger-Müller, 4 distinct stages are defining the different levels of cellulite conditions which can be assessed by a pinch test

Grade 0=no dimples at pinching, grade 1=dimples visible when pinched, grade 2=dimples visible without pinching of skin, but only when standing, grade 3=spontaneous dimpling without pressing of skin in standing and lying.

Cellulite affects women more frequently than men. Cellulite poses, depending on the degree, a strong psychological problem and stress to the affected women.

Cellulite has nothing to do with the dermatological condition known as cellulitis, which is a deep cutaneous inflammation of bacterial origin.

There are numerous treatments for cellulite, including topical, surgical, laser and lymph drainage therapies. However, they are expensive and often ineffective. Therefore, there is a need for compounds that do not have the disadvantage of the products on the market.

One feature of cellulite is excessive accumulation and differentiation of adipocytes in the thighs. This is accompanied by lipogenesis or reduced lipolysis. Therefore, one possible aim of using active substances consists in influencing adipocyte differentiation and concomitantly reducing lipogenesis or activating lipolysis.

We have now surprisingly found that a composition containing at least one compound selected from the group consisting of (−)-epigallocatechin gallate, resveratrol, n-3 polyunsaturated fatty acids, rose hip extract/concentrate, olive polyphenols such as hydroxytyrosol; lycopene, lutein, β-cryptoxanthin, zeaxanthin and derivatives thereof, has an effect in alleviating cellulite when supplied orally to a subject in need thereof. The advantage of such a composition, especially if several compounds of the group are present, is, that it can interfere with several different mechanisms as mentioned above at one time and from inside the body or from outside when applied orally, i.e it reaches the active site of the pathological process. Preferably the compound for such a composition is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof.

Therefore, one object of the present invention is the use of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof, for the treatment of cellulite. “The treatment of cellulite” encompasses the treatment of all 4 grades of cellulite according to Nürnberger-Müller. Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-combinations” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention the following 5-compounds combination is used; epigallocatechin 3-gallate, genistein, resveratrol, β-carotene and a rose hip extract/concentrate. In another preferred embodiment of the present invention the used composition/compound/combination does essentially not contain one of the following compounds: theanine, daidzein and theophylline. Further compounds that are essentially not present in preferred compositions/combinations of the present invention are cited below under “Preferred embodiments of the present invention”.

Another object of the present invention is the use of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof, for the prevention of the development of mild cellulite. “Prevention of the development of mild cellulite” means that female humans that had so far no cellulite are maintained in their healthy status with respect to their skin, i.e. that by use of at least one of the compounds as cited above it may be prevented that such female humans develop a mild/moderate cellulite. Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-combinations” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention the following 5-compounds combination is used: epigallocatechin 3-gallate, genistein, resveratrol, β-carotene and a rose hip extract/concentrate. In another preferred embodiment of the present invention the used composition/compound/combination does essentially not contain one of the following compounds: theanine, daidzein and theophylline. Further compounds that are essentially not present in preferred compositions/combinations of the present invention are cited below under “Preferred embodiments of the present invention”.

A further object of the present invention is the use of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof, for the prevention of the progression of mild cellulite to severe cellulite. Thus, it may be prevented that cellulite of grade 0 or 1 (mild/moderate cellulite) progresses to cellulite of grade 2 or 3 (severe cellulite). Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-combinations” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention the following 5-compounds combination is used: epigallocatechin 3-gallate, genistein, resveratrol, β-carotene and a rose hip extract/concentrate. In another preferred embodiment of the present invention the used composition/compound/combination does essentially not contain one of the following compounds: theanine, daidzein and theophylline. Further compounds that are essentially not present in preferred compositions/combinations of the present invention are cited below under “Preferred embodiments of the present invention”.

A further object of the present invention is also the use of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof, for smoothening the micro relief of the skin. “Smoothening the micro relief of the skin (the skin surface)” encompasses the reduction and resolving of dimpling, and the reduction of the skin's orange peel like and lumpy-bumpy appearance. It also encompasses the maintenance of a smooth and firm skin, especially in the problem areas. Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-combinations” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention the following 5-compounds combination is used: epigallocatechin 3-gallate, genistein, resveratrol, β-carotene and a rose hip extract/concentrate. In another preferred embodiment of the present invention the used composition/compound/combination does essentially not contain one of the following compounds: theanine, daidzein and theophylline. Further compounds that are essentially not present in preferred compositions/combinations of the present invention are cited below under “Preferred embodiments of the present invention”.

A still further object of the present invention is the use of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof, for increasing the tensile properties of the skin. “Maintaining the tensile properties of the skin” means maintaining the skin elasticity. Thus, the skin maintains soft, exhibits healthy hydration and does not get mattress-like appearance. “Increasing the tensile properties of the skin” means increasing the skin elasticity. Thus, the skin gets softer and exhibits improved hydration and looses its mattress-like appearance. Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-combinations” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention the following 5-compounds combination is used: epigallocatechin 3-gallate, genistein, resveratrol, β-carotene and a rose hip extract/concentrate. In another preferred embodiment of the present invention the used composition/compound/combination does essentially not contain one of the following compounds: theanine, daidzein and theophylline. Further compounds that are essentially not present in preferred compositions/combinations of the present invention are cited below under “Preferred embodiments of the present invention”.

Another object of the present invention is the use of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof, for reducing the fat mass and the circumference at the hips and thighs. Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-combinations” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention the following 5-compounds combination is used: epigallocatechin 3-gallate, genistein, resveratrol, β-carotene and a rose hip extract/concentrate. In another preferred embodiment of the present invention the used composition/compound/combination does essentially not contain one of the following compounds: theanine, daidzein and theophylline. Further compounds that are essentially not present in preferred compositions/combinations of the present invention are cited below under “Preferred embodiments of the present invention”.

For the uses listed above neither a combination of (−)-epigallocatechin gallate with docosahexaenoic acid nor a combination of (−)-epigallocatechin gallate with eicosapentaenoic acid is preferred.

The compounds as listed above and below, thus, may reduce the appearance of cellulite; they may work deep down to actively improve the look of problem areas (hips, thighs, bottom/buttock) to measurably slim thighs; they may smooth away the appearance of cellulite; they may help the skin to stay dimple free; they may reduce cellulite; they may tone, firm and hydrate the skin; they may strengthen the skin structure through intense hydration, even in areas most prone to loss of firmness; they may make the skin firmer and improve its tone; they may reduce cellulite; they may be useful for body firming and lifting; they may aim at reducing cellulite and firming the skin texture to give a slimmer and smoother silhouette; they may help stimulate collagen fibres; they may encourage elimination of toxins to restore skin radiance and/or they may dissolve fatty cells, promote circulation and smooth cellulite. Thus, the present invention also refers to these uses.

The group of compounds used for the uses/applications of the present invention are now described in more detail:

### (−)-Epigallocatechin Gallate and Derivatives Thereof

The term “(−)-epigallocatechin gallate” (EGCG) encompasses also green tea extracts containing (−)-EGCG as well as (−)-EGCG derivatives such as pharmaceutically acceptable salts.

An especially suitable (−)-EGCG is e.g. Teavigo (a green tea extract containing ≧94% of EGCG), commercially available from DSM Nutritional Products Ltd, Kaiseraugst, Switzerland, as well as Teavigo TG (Tablet Grade) (a green tea extract containing ca. 88% of EGCG admixed with ca. 3% of pectin).

A preferred alternative for (−)-epigallocatechin gallate is a green tea fraction comprising at least 91.7 weight-% of (−)-epigallocatechin gallate (EGCG) and at most 1.43 weight-% of caffeine, especially a green tea fraction comprising from 91.7 to 97.13 weight-% of EGCG, from 0 to 3.15 weight-% of epicatechin (EC), from 0 to 3.1 weight-% of catechin, from 0.2 to 1.52 weight-% of gallocatechin gallate (GCG), from 0.38 to 4.62 weight-% of epicatechin gallate (ECG) and from 0 to 1.43 weight-% of caffeine.

### Resveratrol and Derivatives Thereof

The term “resveratrol” as used herein comprises a derivative, metabolite or analogue thereof. The carbon-carbon double bond may be trans or cis and includes cis/trans mixtures. Etherified or esterified hydroxy groups may be derived from unsubstituted or substituted, straight or branched chain alkyl groups having 1 to 26 carbon atoms or from unsubstituted or substituted, straight or branched chain aliphatic, araliphatic or aromatic carboxylic acids having 1 to 26 carbon atoms. Etherified hydroxy groups may further be glycoside groups and esterified hydroxy groups may further be glucuronide or sulfate groups. Of primary interest for the purposes of the invention is (trans)-resveratrol.

### n-3 Polyunsaturated Fatty Acids and Derivatives Thereof

Suitable derivatives are the ethyl esters of these acids as well as their mono-, di- and tri-glycerides. Triglycerides of n-3 polyunsaturated fatty acids are especially preferred. Hereby mostly 3 different n-3 polyunsaturated fatty acids are esterified with the glycerin. These triglycerides may also contain partly saturated fatty acids. Examples of such n-3 polyunsaturated acids (PUFAs) are eicosapenta-5,8,11,14,17-enoic acid (EPA) and docosahexa-4,7,10,13,16,19-enoic acid (DHA). In one embodiment of the present invention triglycerides are used, whereby 30% of the fatty acid part are n-3 fatty acids and of these 25% are long-chain polyunsaturated fatty acids. In a further embodiment commercially available ROPUFA® ‘30’ n-3 Food Oil (DSM Nutritional Products Ltd, Kaiseraugst, Switzerland) is used.

Alternatively other polyunsaturated fatty acids (omega-3 fatty acids; omega-6 fatty acids) and/or their derivatives may be used.

### Rose Hip Extract/Concentrate and Derivatives Thereof

Examples of rosehip extract include dried rosehip extract, a lipophilic extract of rosehip (including rosehip oil, rosehip seed oil or a fractionated lipophilic extract) or a hydrophilic extract of rosehips (a rosehip extract comprising watersoluble parts of rosehips, such as for instance polysaccharides), as well as rosehip concentrate/powder (dried and ground rose hips).

Rosehip extract may be obtained from rosehip, the fruit, petals and/or seeds from wild rose bushes, such as Rosa canina (“dog rose-hip”), Rosa gallica, Rosa condita, Rosa rugosa, Rosa hugonis, Rosa nitida, Rosa pendulina, Rosa pimpinellifolia, and Rosa sericea. Preferably, rosehip extract is prepared from the fruit (i.e. the rosehip).

Rosehip extract is a natural substance and hence its exact composition may vary somewhat. The hydrophilic extract preferably has a high vitamin C content in the range of between 0.6 and 1.5 mg per g, as well as other vitamins and minerals. An example of a rosehip extract is that produced in accordance with the description in U.S. Pat. No. 6,024,960. Of course, the quantities of the specific vitamins and minerals may vary by species or through use of different extraction/concentration methods. An exemplary rosehip extract is shown in Table I (page 4 and 5) of WO 02/342274, however the invention is not limited thereto.

Dried rosehip concentrate can be obtained from dried and milled rosehips that are reduced to powder. A rosehip concentrate may for example be obtained in the following method: Rosehips are harvested in a generally known manner when the hips are fully ripe. Rosehip can be obtained from any of the multiple species of plants that belong to the Rosa family, for example from rosehips from wild rose bushes, such as Rosa canina (“dog rose-hip”), Rosa gallica, Rosa condita, Rosa rugosa, Rosa hugonis, Rosa nitida, Rosa pendulina, Rosa pimpinellifolia, and Rosa sericea, more preferably hips of the Rosa canina are used. After the hips are harvested, the hips are chopped into pieces. If further processing is delayed, the hips may be frozen for storage. In any event, the next step is to dry the chopped rosehips to a water content of about at most 5% by weight. Preferably, the drying is conducted in such a way that the vitamin content of the rosehips is preserved, for example by drying the rosehips at a temperature below 50° C. with air and by avoiding sunlight.

The dried and chopped rosehips may then be passed through a separation step in which extraneous matter, for instance nuts, hairs etc., that may have accompanied the rosehips during harvesting is removed. The remaining fruit flesh is then crushed in a grinding mill. Preferably, the obtained material has a particle size of below 1 mm, more preferably between 0.1 and 0.5 mm.

Therefore, a process for obtaining a dried and milled rosehip concentrate may comprise the following steps:
a) harvesting rosehips when the hips are fully ripe
b) chopping the harvested rosehips into pieces
c) drying the chopped rosehips to a water content of at most 5% by weight
d) optionally passing the dried and chopped rosehips through a separation step in which extraneous matter is removed
e) optionally the fruit flesh and the seeds may be separated and extracted separately
f) crushing the remaining material in a grinding mill to a particle size of below 1 mm.

A lipophilic extract of rosehips or a hydrophilic extract may be obtained by methods known to the person skilled in the art. For example, an extract may for example be obtained by extraction from dried and milled rosehips (for example as obtained by the method as described above) through solvent extraction or SFC extraction, using organic or inorganic solvents, examples of which include supercritical CO2, hexane, dichloromethane, ethanol and water. After extraction, the obtained extract may be evaporated to dryness to recover an extract fraction.

Preferably, the extract (fraction) comprises at least cyanidin-3-o-glucoside, cyanidin-3-o-rutinoside or glycosides of diacylglycerol such as 3-.beta.-D-galactopyranosyloxy-2-(octadeca-9Z,12Z,15Z-trienoyloxy)propanyl octadeca-9Z,12Z,15Z-trienoate (GOPO), in particular at least GOPO.

### Hydroxytyrosol and Derivatives Thereof

Hydroxytyrosol may be of synthetic origin or it may be obtained together with other water-soluble polyphenols such as tyrosol and oleuropein from extraction of olive leaves, olive fruits and vegetation water of olive oil production.

Examples of references that deal with the extraction of oleuropein and/or hydroxytyrosol from olive leaves are WO02/18310, US 2002/0198415, WO2004/005228, U.S. Pat. No. 6,416,808 and US 2002/0058078 which disclose a method for acidic hydrolysis of olive vegetation water for 2 to 12 months until at least 90% of the present oleuropein has been converted. A method of extraction of phenolic compounds from olives, olive pulps, olive oil and oil mill waste water is described by Usana Inc. U.S. Pat. No. 6,361,803 and WO01/45514 and in US 2002/0004077. EP-A 1 582 512 describes an extraction of hydroxytyrosol from olive leaves. A method for obtaining hydroxytyrosol and/or oleuropein from the vegetation water of de-pitted olives is disclosed in US 2004/0039066 A1 in paragraphs [0080]-[0091].

Derivatives may be esters as well as physiologically/pharmaceutically acceptable salts.

### Lycopene and Derivatives Thereof

The term “lycopene” includes all-E and Z-stereoisomers. Alternatively a tomato extract which contains high amounts of lycopene could also be used.

### Lutein and Derivatives Thereof

The term “lutein” includes all-E and Z-stereoisomers. Suitable derivatives are e.g. its mono- and di-esters, preferably esters of saturated alkanoic acids such as acetic, propionic, laurinic, myristinic, palmitic, stearic and succinic acid, esters of mono-unsaturated fatty acids such as oleic acid, and poly-unsaturated fatty acids such as linolic, linoleic, pentaenoic, docosahexaenoic and arachidonic acid, and mixtures thereof.

### β-Cryptoxanthin and Derivatives Thereof

The term “β-cryptoxanthin” includes all-E and Z-stereoisomers. Suitable derivatives are e.g. its mono- and di-esters, preferably esters of saturated alkanoic acids such as acetic, propionic, laurinic, myristinic, palmitic, stearic and succinic acid, esters of mono-unsaturated fatty acids such as oleic acid, and poly-unsaturated fatty acids such as linolic, linoleic, pentaenoic, docosahexaenoic and arachidonic acid, and mixtures thereof.

### Zeaxanthin and Derivatives Thereof

The term “zeaxanthin” includes all-E and Z-stereoisomers.

### Further Active Ingredients

Further compounds that may be used in combination of at least one compound selected from the group consisting of (−)-epigallocatechin gallate, resveratrol, n-3 polyunsaturated fatty acids, rose hip extract/concentrate, hydroxytyrosol, lycopene, lutein, β-cryptoxanthin, zeaxanthin and derivatives thereof, are:

isoflavones such as genistein; fat-soluble vitamins such as Vitamin A, Vitamin E, Vitamin D, Vitamin K and derivatives thereof such as the acetates and palmitates; carotenoids with pro-vitamin A activity such as β-carotene and α-carotene (whereby β-carotene is preferred); water-soluble vitamins such as Vitamin C and the B-vitamines and derivatives thereof (especially preferred are Vitamin C and Vitamin B12); biotin and derivatives thereof; ubiquinones such as coenzyme Q10 (CoQ-10).

The use of a 2-compounds-combination of (−)-epigallocatechin gallate and genistein is not preferred.

Further active ingredients are horse chestnut extract (rutin), centella asiatica extract, caffeine and pycnogenol, as well as Hexamethoxyfiavone, Mangostin, 6,8-Diprenylnaringenin; from sage or rosemary: Camosol, rosmanol or 7-Epimer, 7-methylrosmanol or 7-Epimer, 20-Deoxo-carnosol, Sageone, Camosic acid, 8,11,13-Abietatriene-11,12,20-triol; from Cashew: Cardoltriene, Cardoldiene, (15:3)-Anacardic acid; from Mango ginger: Zerumin A, From blue fenugreek, Cannabigerolic acid monomethyl ether; from Garcinia cambogia: Guttiferone A; from magnolia bark: Honokiol, Magnolol; from Hops: 6-hydroxylupulone, 6-Hydroxycolupulone; from Amomum melegueta (grains of paradise): [4]-Gingerdiol, 6-Hydroxy-[4]-Ginger-4-one, [6]-Gingerdiol, [6]-Gingerol, Shorbic acid; from Arctium lappa: 1,5-Di-O-caffeoyl-3-β-succinylquinic acid, 1,5-Di-O-caffeoyl-3,4-di-O-succinylquinic acid.

In an embodiment of the present invention (−)-epigallocatechin gallate is used as the sole active ingredient in the uses/applications of the present invention. In another embodiment of the present invention resveratrol is used as the sole active ingredient in the uses/applications of the present invention. In a further embodiment of the present invention a n-3 polyunsaturated fatty acid and/or a derivative thereof is/are used as the sole active ingredient/s in the uses/applications of the present invention. In a further embodiment of the present invention a rose hip extract/concentrate is used as the sole active ingredient in the uses/applications of the present invention. In a further embodiment of the present invention olive polyphenols such as hydroxytyrosol is used as the sole active ingredient in the uses/applications of the present invention. In another embodiment of the present invention lycopene is used as the sole active ingredient in the uses/applications of the present invention. In a further embodiment of the present invention lutein is used as the sole active ingredient in the uses/applications of the present invention. In another embodiment of the present invention β-cryptoxanthin is used as the sole active ingredient in the uses/applications of the present invention. In a further embodiment of the present invention zeaxanthin is used as the sole active ingredient in the uses/applications of the present invention.

In a further embodiment of the present invention biotin is used as the sole active ingredient in the uses/applications of the present invention. In a further embodiment of the present invention Vitamin E is used as the sole active ingredient in the uses/applications of the present invention. In a further embodiment of the present invention Vitamin C is used as the sole active ingredient in the uses/applications of the present invention. In a further embodiment of the present invention Vitamin B12 is used as the sole active ingredient in the uses/applications of the present invention. In a further embodiment of the present invention CoQ-10 is used as the sole active ingredient in the uses/applications of the present invention. In a further embodiment of the present invention β-carotene is used as the sole active ingredient in the uses/applications of the present invention.

In embodiments of the present invention the following combinations of active ingredients (as defined above) are present:

(−)-Epigallocatechin gallate and resveratrol (especially preferred);
(−)-epigallocatechin gallate and a n-3/n-6 polyunsaturated fatty acid (derivative) (not preferred);
(−)-epigallocatechin gallate and rose hip extract/concentrate;
(−)-epigallocatechin gallate and an olive polyphenol such as hydroxytyrosol;
(−)-epigallocatechin gallate and lycopene;
(−)-epigallocatechin gallate and lutein;
(−)-epigallocatechin gallate and β-cryptoxanthin;
(−)-epigallocatechin gallate and zeaxanthin;
(−)-epigallocatechin gallate and genistein (not preferred);
(−)-epigallocatechin gallate and biotin;
(−)-epigallocatechin gallate and vitamin C;
(−)-epigallocatechin gallate and Vitamin E;
(−)-epigallocatechin gallate and β-carotene;
(−)-epigallocatechin gallate and CoQ-10;
(−)-epigallocatechin gallate and vitamin B12;
resveratrol and a n-3/n-6 polyunsaturated fatty acid (derivative);
resveratrol and rose hip extract/concentrate (especially preferred);
resveratrol and an olive polyphenol such as hydroxytyrosol;
resveratrol and lycopene;
resveratrol and lutein;
resveratrol and β-cryptoxanthin;
resveratrol and zeaxanthin;
resveratrol and genistein (especially preferred);
resveratrol and biotin;
resveratrol and vitamin C;
resveratrol and Vitamin E;
resveratrol and β-carotene;
resveratrol and CoQ-10;
resveratrol and vitamin B12;
a n-3/n-6 polyunsaturated fatty acid (derivative) and rose hip extract/concentrate;
a n-3/n-6 polyunsaturated fatty acid (derivative) and an olive polyphenol such as hydroxytyrosol (especially preferred);
a n-3/n-6 polyunsaturated fatty acid (derivative) and lycopene;
a n-3/n-6 polyunsaturated fatty acid (derivative) and lutein;
a n-3/n-6 polyunsaturated fatty acid (derivative) and β-cryptoxanthin;
a n-3/n-6 polyunsaturated fatty acid (derivative) and zeaxanthin;
a n-3/n-6 polyunsaturated fatty acid (derivative) and genistein;
a n-3/n-6 polyunsaturated fatty acid (derivative) and biotin;
a n-3/n-6 polyunsaturated fatty acid (derivative) and vitamin C;
a n-3/n-6 polyunsaturated fatty acid (derivative) and Vitamin E;
a n-3/n-6 polyunsaturated fatty acid (derivative) and β-carotene;
a n-3/n-6 polyunsaturated fatty acid (derivative) and CoQ-10;
a n-3/n-6 polyunsaturated fatty acid (derivative) and vitamin B12;
a rose hip extract/concentrate and an olive polyphenol such as hydroxytyrosol (especially preferred);
a rose hip extract/concentrate and lycopene;
a rose hip extract/concentrate and lutein;
a rose hip extract/concentrate and β-cryptoxanthin;
a rose hip extract/concentrate and zeaxanthin;
a rose hip extract/concentrate and genistein;
a rose hip extract/concentrate and biotin;
a rose hip extract/concentrate and vitamin C;
a rose hip extract/concentrate and Vitamin E;
a rose hip extract/concentrate and β-carotene (especially preferred);
a rose hip extract/concentrate and CoQ-10;
a rose hip extract/concentrate and vitamin B12;
an olive polyphenol such as hydroxytyrosol and lycopene;
an olive polyphenol such as hydroxytyrosol and lutein;
an olive polyphenol such as hydroxytyrosol and β-cryptoxanthin;
an olive polyphenol such as hydroxytyrosol and zeaxanthin;
an olive polyphenol such as hydroxytyrosol and genistein;
an olive polyphenol such as hydroxytyrosol and biotin;
an olive polyphenol such as hydroxytyrosol and vitamin C;
an olive polyphenol such as hydroxytyrosol and Vitamin E;
an olive polyphenol such as hydroxytyrosol and β-carotene;
an olive polyphenol such as hydroxytyrosol and CoQ-10;
an olive polyphenol such as hydroxytyrosol and vitamin B12;
lycopene and lutein;
lycopene and β-cryptoxanthin;
lycopene and zeaxanthin;
lycopene and genistein;
lycopene and biotin;
lycopene and vitamin C;
lycopene and Vitamin E;
lycopene and β-carotene;
lycopene and CoQ-10;
lycopene and vitamin B12;
lutein and β-cryptoxanthin;
lutein and zeaxanthin;
lutein and genistein;
lutein and biotin;
lutein and vitamin C;
lutein and Vitamin E;
lutein and β-carotene;
lutein and CoQ-10;
lutein and vitamin B12;
β-cryptoxanthin and zeaxanthin;
β-cryptoxanthin and genistein;
β-cryptoxanthin and biotin;
β-cryptoxanthin and vitamin C;
β-cryptoxanthin and Vitamin E;
β-cryptoxanthin and β-carotene;
β-cryptoxanthin and CoQ-10;
β-cryptoxanthin and vitamin B12;
zeaxanthin and genistein;
zeaxanthin and biotin;
zeaxanthin and vitamin C;
zeaxanthin and Vitamin E;
zeaxanthin and β-carotene;
zeaxanthin and CoQ-10;
zeaxanthin and vitamin B12;
genistein and biotin;
genistein and vitamin C;
genistein and Vitamin E;
genistein and β-carotene (especially preferred);
genistein and CoQ-10;
genistein and vitamin B12;
biotin and vitamin C;
biotin and Vitamin E;
biotin and β-carotene;
biotin and CoQ-10;
biotin and vitamin B12;
vitamin C and Vitamin E;
vitamin C and β-carotene;
vitamin C and CoQ-10;
vitamin C and vitamin B12;
vitamin E and β-carotene;
vitamin E and CoQ-10;
vitamin E and vitamin B12;
β-carotene and CoQ-10;
β-carotene and vitamin B12;
CoQ-10 and vitamin B12;
(−)-epigallocatechin gallate, genistein and resveratrol;
(−)-epigallocatechin gallate, genistein and β-carotene;
(−)-epigallocatechin gallate, resveratrol and β-carotene;
(−)-epigallocatechin gallate, resveratrol and a n-3/n-6 polyunsaturated fatty acid (derivative);
(−)-epigallocatechin gallate, β-carotene and a n-3/n-6 polyunsaturated fatty acid (derivative);
(−)-epigallocatechin gallate, genistein and a n-3/n-6 polyunsaturated fatty acid (derivative);
genistein, resveratrol and β-carotene;
resveratrol, genistein and a n-3/n-6 polyunsaturated fatty acid (derivative);
β-carotene, genistein and a n-3/n-6 polyunsaturated fatty acid (derivative);
β-carotene, resveratrol and a n-3/n-6 polyunsaturated fatty acid (derivative);
genistein, resveratrol, β-carotene and a n-3/n-6 polyunsaturated fatty acid (derivative);
(−)-epigallocatechin gallate, resveratrol, β-carotene and a n-3/n-6 polyunsaturated fatty acid (derivative);
(−)-epigallocatechin gallate, genistein, β-carotene and a n-3/n-6 polyunsaturated fatty acid (derivative);
(−)-epigallocatechin gallate, genistein, resveratrol and a n-3/n-6 polyunsaturated fatty acid (derivative);
(−)-epigallocatechin gallate, genistein, resveratrol and β-carotene;
(−)-epigallocatechin gallate, genistein, resveratrol, β-carotene and a n-3/n-6 polyunsaturated fatty acid (derivative) (especially preferred);
(−)-epigallocatechin gallate, genistein, resveratrol, β-carotene and a rose hip extract/concentrate (especially preferred).

In the same way (at least) one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof, (preferably (at least) one compound selected from the group consisting of resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof) may be combined with (at least) one compound selected from the group consisting of horse chestnut extract (rutin), centella asiatica extract, caffeine, pycnogenol, Hexamethoxyflavone, Mangostin, 6,8-Diprenylnaringenin; Carnosol, rosmanol or 7-Epimer, 7-methylrosmanol or 7-Epimer, 20-Deoxo-camosol, Sageone, Carnosic acid, 8,11,13-Abietatriene-11,12,20-triol; Cardoltriene, Cardoldiene, (15:3)-Anacardic acid; Zerumin A, From blue fenugreek, Cannabigerolic acid monomethyl ether; Guttiferone A; Honokiol, Magnolol; from Hops: 6-hydroxylupulone, 6-Hydroxycolupulone; [4]-Gingerdiol, 6-Hydroxy-[4]-Ginger-4-one, [6]-Gingerdiol, [6]-Gingerol, Shorbic acid; 1,5-Di-O-caffeoyl-3-O-succinylquinic acid and 1,5-Di-O-caffeoyl-3,4-di-O-succinylquinic acid.

In further embodiments of the present invention these combinations of active ingredients cited above are the sole active ingredients present in compositions that are used in uses/applications of the present invention as cited above,

### PREFERRED EMBODIMENTS OF THE PRESENT INVENTION

Preferred embodiments of the compositions of the present invention do (essentially) not comprise one of the following compounds: theanine, daidzein and theophylline.

More preferred embodiments of the compositions of the present invention do (essentially) not comprise one of the following compounds: theanine, daidzein, theophylline, norepinephrine, caffeine, (L-)carnitine, theobromine and aminophylline.

Even more preferred embodiments of the compositions of the present invention do essentially not comprise any of the following compounds: theanine, daidzein and theophylline.

Most preferred embodiments of the compositions of the present invention do essentially not comprise any of the following compounds: theanine, daidzein, theophylline, norepinephrine, caffeine, (L-)carnitine, theobromine and aminophylline.

These preferences apply to the compositions/combinations as listed above, as well as to the ones as listed below.

The term “does essentially not comprise” means that none of the compounds as listed above are additionally added to the compositions of the present invention. If compositions containing plant extracts are used and the plant extracts contain only traces (preferably 0.000001 weight-% based on the total weight of the composition) of such compounds as listed above, such compositions would still be in the scope of the present invention.

It is especially preferred to administer the compositions, compounds and/or combinations of active ingredients of the present invention orally to female humans.

Especially preferred of the combinations of compounds named above are the following ones:

resveratrol+rose hip extract/concentrate;
resveratrol+EGCG;
resveratrol+genistein;
hydroxytyrosol+rose hip extract/concentrate;
hydroxytyrosol+docosahexaenoic acid (DHA);
β-carotene+genistein;
β-carotene+rose hip extract/concentrate
β-carotene+EGCG+resveratrol+genistein+rose hip extract/concentrate.

The amounts in which the compounds may be administered are listed below.

Thus, the present invention is directed to the use of a composition comprising resveratrol and a rose hip extract/concentrate for the treatment of cellulite in female humans, for the prevention of the development of mild cellulite in female humans, for the prevention of the progression of mild cellulite to severe cellulite in female humans, for smoothening the micro relief of the skin of female humans, for maintaining or increasing the tensile properties of the skin of female humans, for reducing the fat mass and the circumference at the hips and thighs of female humans, for the maintenance of a smooth and firm skin of female humans and/or the beautification of the silhouette/bodyshape of female humans.

Thus, the present invention is directed to the use of a composition comprising resveratrol and EGCG for the treatment of cellulite in female humans, for the prevention of the development of mild cellulite in female humans, for the prevention of the progression of mild cellulite to severe cellulite in female humans, for smoothening the micro relief of the skin of female humans, for maintaining or increasing the tensile properties of the skin of female humans, for reducing the fat mass and the circumference at the hips and thighs of female humans, for the maintenance of a smooth and firm skin of female humans and/or the beautification of the silhouette/bodyshape of female humans.

Thus, the present invention is directed to the use of a composition comprising resveratrol and genistein for the treatment of cellulite in female humans, for the prevention of the development of mild cellulite in female humans, for the prevention of the progression of mild cellulite to severe cellulite in female humans, for smoothening the micro relief of the skin of female humans, for maintaining or increasing the tensile properties of the skin of female humans, for reducing the fat mass and the circumference at the hips and thighs of female humans, for the maintenance of a smooth and firm skin of female humans and/or the beautification of the silhouette/bodyshape of female humans.

Thus, the present invention is directed to the use of a composition comprising hydroxytyrosol and a rose hip extract/concentrate for the treatment of cellulite in female humans, for the prevention of the development of mild cellulite in female humans, for the prevention of the progression of mild cellulite to severe cellulite in female humans, for smoothening the micro relief of the skin of female humans, for maintaining or increasing the tensile properties of the skin of female humans, for reducing the fat mass and the circumference at the hips and thighs of female humans, for the maintenance of a smooth and firm skin of female humans and/or the beautification of the silhouette/bodyshape of female humans.

Thus, the present invention is directed to the use of a composition comprising hydroxytyrosol and docosahexaenoic acid (DHA) for the treatment of cellulite in female humans, for the prevention of the development of mild cellulite in female humans, for the prevention of the progression of mild cellulite to severe cellulite in female humans, for smoothening the micro relief of the skin of female humans, for maintaining or increasing the tensile properties of the skin of female humans, for reducing the fat mass and the circumference at the hips and thighs of female humans, for the maintenance of a smooth and firm skin of female humans and/or the beautification of the silhouette/bodyshape of female humans.

Thus, the present invention is directed to the use of a composition comprising β-carotene and genistein for the treatment of cellulite in female humans, for the prevention of the development of mild cellulite in female humans, for the prevention of the progression of mild cellulite to severe cellulite in female humans, for smoothening the micro relief of the skin of female humans, for maintaining or increasing the tensile properties of the skin of female humans, for reducing the fat mass and the circumference at the hips and thighs of female humans, for the maintenance of a smooth and firm skin of female humans and/or the beautification of the silhouette/bodyshape of female humans.

Thus, the present invention is directed to the use of a composition comprising β-carotene and a rose hip extract/concentrate for the treatment of cellulite in female humans, for the prevention of the development of mild cellulite in female humans, for the prevention of the progression of mild cellulite to severe cellulite in female humans, for smoothening the micro relief of the skin of female humans, for maintaining or increasing the tensile properties of the skin of female humans, for reducing the fat mass and the circumference at the hips and thighs of female humans, for the maintenance of a smooth and firm skin of female humans and/or the beautification of the silhouette/bodyshape of female humans.

Thus, the present invention is directed to the use of a composition comprising β-carotene and EGCG and resveratrol and genistein and a rose hip extract/concentrate for the treatment of cellulite in female humans, for the prevention of the development of mild cellulite in female humans, for the prevention of the progression of mild cellulite to severe cellulite in female humans, for smoothening the micro relief of the skin of female humans, for maintaining or increasing the tensile properties of the skin of female humans, for reducing the fat mass and the circumference at the hips and thighs of female humans, for the maintenance of a smooth and firm skin of female humans and/or the beautification of the silhouette/bodyshape of female humans.

In further embodiments of the present invention these combinations of active ingredients cited above are the sole active ingredients present in compositions that are used in uses/applications of the present invention as cited above.

### Dietary, Cosmetic, Dermatological and Pharmaceutical Compositions of the Present Invention

A dietary, cosmetic, dermatological or pharmaceutical composition containing at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof. Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-compositions” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention the following 5-compounds composition is used: epigallocatechin 3-gallate, genistein, resveratrol, β-carotene and a rose hip extract/concentrate. In another preferred embodiment of the present invention the used composition/compound/combination does essentially not contain one of the following compounds: theanine, daidzein and theophylline. Further compounds that are essentially not present in preferred compositions/combinations of the present invention are cited above under “Preferred embodiments of the present invention”.

In a special embodiment of the present invention the dietary composition may be in form of food such as dairy products (yoghurts), in form of fortified food such as cereal bars and bakery items such as cakes and cookies, in form of dietary supplements such as tablets, pills, granules, dragées, capsules, instant drinks and effervescent formulations, in form of non-alcoholic drinks such as soft drinks, sport drinks, fruit juices, lemonades, teas and milk based drinks, in form of liquid food such as soups and dairy products (muesli drinks). Such dietary compositions may be used for the treatment of cellulite, for the prevention of the development of mild cellulite, for the prevention of the progression of mild cellulite to severe cellulite, for smoothening the micro relief of the skin, for increasing the tensile properties of the skin and for reducing the fat mass and the circumference at the hips and thighs, as well as for the other uses/applications of the present invention as cited above.

Thus, the present invention is also directed to a dietary, cosmetic or dermatological composition for the treatment of cellulite in female humans, for the prevention of the development of mild cellulite in female humans, for the prevention of the progression of mild cellulite to severe cellulite in female humans, for smoothening the micro relief of the skin of female humans, for maintaining or increasing the tensile properties of the skin of female humans, for reducing the fat mass and the circumference at the hips and thighs of female humans, for the maintenance of a smooth and firm skin of female humans and/or for the beautification of the silhouette/bodyshape of female humans, said composition containing at least one compound selected from the group consisting of (−)-epigallo-catechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof. Here the same preferences concerning the selected compounds and 2-compounds- or 5-compounds-combinations as mentioned above apply.

### METHODS OF THE PRESENT INVENTION

The present invention is directed to a cosmetic or medical method for the treatment of cellulite by administering to a subject in need thereof an effective amount of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof. Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-combinations” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention one of the following two 5-compounds combination is used: epigallocatechin 3-gallate, genistein, resveratrol, β-carotene and a rose hip extract/concentrate; or epigallocatechin 3-gallate, genistein, resveratrol, β-carotene and a n-3/n-6 polyunsaturated fatty acid (derivative). In another preferred embodiment of the present invention the used composition/compound/combination does essentially not contain one of the following compounds: theanine, daidzein and theophylline. Further compounds that are essentially not present in preferred compositions/combinations of the present invention are cited above under “Preferred embodiments of the present invention”.

In a further preferred cosmetic or medical method for the treatment of cellulite an effective amount of at least one of the active, ingredients selected from the group consisting of horse chestnut extract (rutin), centella asiatica extract, genistein and pycnogenol is administered orally subsequently, simultaneously or in advance. Preferably the further active ingredient is selected from the group consisting of horse chestnut extract (rutin), centella asiatica extract and pycnogenol.

The present invention is further directed to a cosmetic or medical method for the prevention of the development of mild cellulite by administering to a subject in need thereof an effective amount of at least one compound selected from the group consisting of (−)-epigallocatechin gallate, resveratrol, n-3 polyunsaturated fatty acids, rose hip extract/concentrate, an olive polyphenol such as hydroxytyrosol, lycopene, lutein, β-cryptoxanthin, zeaxanthin and derivatives thereof. Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-combinations” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention one of the following two 5-compounds combination is used: epigallocatechin 3-gallate, genistein, resveratrol, β-carotene and a rose hip extract/concentrate; or epigallocatechin 3-gallate, genistein, resveratrol, β-carotene and a n-3/n-6 polyunsaturated fatty acid (derivative). In another preferred embodiment of the present invention the used composition/compound/combination does essentially not contain one of the following compounds: theanine, daidzein and theophylline. Further compounds that are essentially not present in preferred compositions/combinations of the present invention are cited above under “Preferred embodiments of the present invention”.

In a further preferred cosmetic or medical method for the prevention of the development of mild cellulite an effective amount of at least one of the active ingredients selected from the group consisting of horse chestnut extract (rutin), centella asiatica extract, genistein and pycnogenol is administered orally subsequently, simultaneously or in advance. Preferably the further active ingredient is selected from the group consisting of horse chestnut extract (rutin), centella asiatica extract and pycnogenol.

Moreover, the present invention is directed to a cosmetic or medical method for the prevention of the progression of mild cellulite to severe cellulite by administering to a subject in need thereof an effective amount of at least one compound selected from the group consisting of (−)-epigallocatechin gallate, resveratrol, n-3 polyunsaturated fatty acids, rose hip extract/concentrate, an olive polyphenol such as hydroxytyrosol, lycopene, lutein, β-cryptoxanthin, zeaxanthin and derivatives thereof. Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-combinations” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention one of the following two 5-compounds combination is used: epigallocatechin 3-gallate, genistein, resveratrol, β-carotene and a rose hip extract/concentrate; or epigallocatechin 3-gallate, genistein, resveratrol, β-carotene and a n-3/n-6 polyunsaturated fatty acid (derivative). In another preferred embodiment of the present invention the used composition/compound/combination does essentially not contain one of the following compounds: theanine, daidzein and theophylline. Further compounds that are essentially not present in preferred compositions/combinations of the present invention are cited above under “Preferred embodiments of the present invention”.

In a further preferred cosmetic or medical method for the prevention of the progression of mild cellulite to severe cellulite an effective amount of at least one of the active ingredients selected from the group consisting of horse chestnut extract (rutin), centella asiatica extract, genistein and pycnogenol is administered orally subsequently, simultaneously or in advance. Preferably the further active ingredient is selected from the group consisting of horse chestnut extract (rutin), centella asiatica extract and pycnogenol.

Another object of the present invention is a cosmetic or medical method for smoothening the micro relief of the skin by administering to a subject in need thereof an effective amount of at least one compound selected from the group consisting of (−)-epigallo-catechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof. Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-combinations” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention one of the following two 5-compounds combination is used: epigallocatechin 3-gallate, genistein, resveratrol, β-carotene and a rose hip extract/concentrate; or epigallocatechin 3-gallate, genistein, resveratrol, β-carotene and a n-3/n-6 polyunsaturated fatty acid (derivative). In another preferred embodiment of the present invention the used composition/compound/combination does essentially not contain one of the following compounds: theanine, daidzein and theophylline. Further compounds that are essentially not present in preferred compositions/combinations of the present invention are cited above under “Preferred embodiments of the present invention”.

In a further preferred cosmetic or medical method for smoothening the micro relief of the skin an effective amount of at least one of the active ingredients selected from the group consisting of horse chestnut extract (rutin), centella asiatica extract, genistein and pycnogenol is administered orally subsequently, simultaneously or in advance. Preferably the further active ingredient is selected from the group consisting of horse chestnut extract (rutin), centella asiatica extract and pycnogenol.

Furthermore, the present invention is directed to a cosmetic or medical method for increasing the tensile properties of the skin by administering to a subject in need thereof an effective amount of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof. Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-combinations” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention one of the following two 5-compounds combination is used: epigallocatechin 3-gallate, genistein, resveratrol, β-carotene and a rose hip extract/concentrate; or epigallocatechin 3-gallate, genistein, resveratrol, β-carotene and a n-3/n-6 polyunsaturated fatty acid (derivative). In another preferred embodiment of the present invention the used composition/compound/combination does essentially not contain one of the following compounds: theanine, daidzein and theophylline. Further compounds that are essentially not present in preferred compositions/combinations of the present invention are cited above under “Preferred embodiments of the present invention”.

In a further preferred cosmetic or medical method for increasing the tensile properties of the skin an effective amount of at least one of the active ingredients selected from the group consisting of horse chestnut extract (rutin), centella asiatica extract, genistein and pycnogenol is administered orally subsequently, simultaneously or in advance. Preferably the further active ingredient is selected from the group consisting of horse chestnut extract (rutin), centella asiatica extract and pycnogenol.

The present invention is also directed to a cosmetic or medical method for reducing the fat mass and the circumference at the hips and thighs by administering to a subject in need thereof an effective amount of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof. Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-combinations” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention one of the following two 5-compounds combination is used: epigallocatechin 3-gallate, genistein, resveratrol, β-carotene and a rose hip extract/concentrate; or epigallocatechin 3-gallate, genistein, resveratrol, β-carotene and a n-3/n-6 polyunsaturated fatty acid (derivative). In another preferred embodiment of the present invention the used composition/compound/combination does essentially not contain one of the following compounds: theanine, daidzein and theophylline. Further compounds that are essentially not present in preferred compositions/combinations of the present invention are cited above under “Preferred embodiments of the present invention”.

In a further preferred cosmetic or medical method for reducing the fat mass and the circumference at the hips and thighs an effective amount of at least one of the active ingredients selected from the group consisting of horse chestnut extract (rutin), centella asiatica extract, genistein and pycnogenol is administered orally subsequently, simultaneously or in advance. Preferably the further active ingredient is selected from the group consisting of horse chestnut extract (rutin), centella asiatica extract and pycnogenol.

The present invention is also directed to a cosmetic method for maintaining a smooth and/or firm skin and/or for beautifying the silhouette/bodyshape by administering to a subject in need thereof an effective amount of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof.

Preferably the compound is selected from the group consisting of resveratrol, n-3 polyunsaturated fatty acids, rose hip extract/concentrate, olive polyphenols such as hydroxytyrosol; lycopene, lutein, β-cryptoxanthin, zeaxanthin and derivatives thereof. More preferably one of the following “two-compounds-combinations” is used: resveratrol and a rose hip extract/concentrate; hydroxytyrosol and a rose hip extract/concentrate; hydroxytyrosol and docosahexaenoic acid; resveratrol and genistein; resveratrol and (−)-epigallocatechin 3-gallate; β-carotene and a rose hip extract/concentrate; β-carotene and genistein. In a further preferred embodiment of the present invention one of the following two 5-compounds combination is used: epigallocatechin 3-gallate, genistein, resveratrol, β-carotene and a rose hip extract/concentrate; or epigallocatechin 3-gallate, genistein, resveratrol, β-carotene and a n-3/n-6 polyunsaturated fatty acid (derivative). In another preferred embodiment of the present invention the used composition/compound/combination does essentially not contain one of the following compounds: theanine, daidzein and theophylline. Further compounds that are essentially not present in preferred compositions/combinations of the present invention are cited above under “Preferred embodiments of the present invention”.

In a further preferred cosmetic or medical method for maintaining a smooth and/or firm skin and/or for beautifying the silhouette/bodyshape an effective amount of at least one of the active ingredients selected from the group consisting of horse chestnut extract (rutin), centella asiatica extract, genistein and pycnogenol is administered orally subsequently, simultaneously or in advance. Preferably the further active ingredient is selected from the group consisting of horse chestnut extract (rutin), centella asiatica extract and pycnogenol.

Furthermore, the present invention is preferably directed to a cosmetic or medical method for the treatment of cellulite, for the prevention of the development of mild cellulite, for the prevention of the progression of mild cellulite to severe cellulite, for smoothening the micro relief of the skin, for maintaining or increasing the tensile properties of the skin, for reducing the fat mass and the circumference at the hips and thighs, for the maintenance of a smooth and firm skin and/or the beautification of the silhouette/bodyshape by administering to a subject in need thereof an effective amount of a composition comprising (−)-epigallocatechin gallate, genistein, resveratrol, β-carotene and a n-3/n-6 polyunsaturated fatty acid (derivative).

The present invention is also preferably directed to a cosmetic or medical method for the treatment of cellulite, for the prevention of the development of mild cellulite, for the prevention of the progression of mild cellulite to severe cellulite, for smoothening the micro relief of the skin, for maintaining or increasing the tensile properties of the skin, for reducing the fat mass and the circumference at the hips and thighs, for the maintenance of a smooth and firm skin and/or the beautification of the silhouette/bodyshape by administering to a subject in need thereof an effective amount of a composition comprising (−)-epigallocatechin gallate, genistein, resveratrol, β-carotene and a rose hip extract/concentrate.

In preferred embodiments of the present invention the effective amount(s) of the active ingredient(s) is/are administered orally.

According to other embodiments of the present invention other active ingredients as listed above, especially at least one selected from the group consisting of biotin, Vitamin E, Vitamin C, Vitamin B12, CoQ-10, β-carotene, horse chestnut extract (esp. rutin), centella asiatica extract and pycnogenol is administered orally subsequently, simultaneously or in advance to an effective amount of at least one of the active ingredients selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof.

The subject in need thereof in the context of the present invention are female humans, especially in the age of from 15 to 70. But the method for smoothening the micro relief of the skin, the method for maintaining or increasing the tensile properties of the skin, the method for maintaining a smooth and firm skin and the method for beautifying the silhouette/bodyshape (or use of the compounds/combinations/compositions for that purpose), i.e. in total a method for improving the physical appearance, may also be applied to male humans, especially in that age.

Any of the methods mentioned above is preferably combined with a method of topically administering to said subject in need thereof at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof. Preferably the at least one compound is selected from the group consisting of resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof.

In further embodiments of the present invention any of the methods mentioned above is combined with a method of stimulating the skin of said subject in need thereof mechanically by massages or by ultrasound or any other method or combination of method known to the person skilled in the art.

The present invention is also directed to the use of any compound/combination or composition according to the present invention for reducing the differentiation of pre-adipocytes to adipocytes in female humans.

The present invention is further directed to the use of any compound/combination or composition according to the present invention for preventing the adipocyte differentiation in female humans.

The effective amounts of the compounds as listed above may lay preferably in the following ranges:

(−)-Epigallocatechin gallate: daily dosage for humans (70 kg person): 50 to 600 mg, preferred daily dosage for humans (70 kg person): 150 to 300 mg.

Resveratrol: daily dosage for humans (70 kg person): 1 to 100 mg, preferred daily dosage for humans (70 kg person): 5 to 50 mg, more preferred daily dosage for humans (70 kg person): 20 to 30 mg.

n-3 polyunsaturated fatty acid (derivative): daily dosage for humans (70 kg person): 500 mg to 5 g for a n-3 polyunsaturated fatty acid triglyceride.

Rose hip extract: The nutraceutical compositions of the present invention preferably comprise rosehip extract in an amount sufficient to administer to a human adult (weighing about 70 kg) a dosage from 20 mg/day based on the weight of dried rosehip concentrate to 30 g/day based on the weight of dried rosehip concentrate, preferably from 2 g/day to 10 g/day based on the weight of dried rosehip concentrate; more preferably from 5 g/day to 5 g/day based on the weight of dried rosehip concentrate.

Thus, if the nutraceutical composition is a food or beverage the amount of a dried rosehip concentrate comprised therein is preferably in the range from about 0.2 g to about 10 g per serving. If the composition is a pharmaceutical composition such composition may for example comprise from 20 mg to 1 g per solid dosage unit, e.g., per capsule or tablet, or for example from 500 mg per daily dose to 6000 mg per daily dose of a liquid formulation.

If instead of dried rosehip concentrate, rosehip oil or an individual rosehip compound is used, the amount used may be derived from the amount of dried rosehip concentrate and finding the optimal dosage is a matter of routine experimentation for the person skilled in the art.

Hydroxytyrosol: daily dosage for humans (70 kg person): 5 to 500 mg, preferred daily dosage for humans (70 kg person): 50 to 100 mg.

Lycopene: For usual applications the daily dosage for humans (usually determined for a 70 kg person) for lutein should not exceed 40 mg, preferably not exceed 25 mg. In some embodiments of the invention the daily dosage for humans (70 kg person) for lutein can be between 0.1 to 40 mg, more preferably between 0.5 to 25 mg.

Lutein: For usual applications the daily dosage for humans (usually determined for a 70 kg person) for lycopene not exceed 60 mg, preferably not exceed 30 mg.

In some embodiments of the invention the daily dosage for humans (70 kg person) for lycopene between 0.1 to 60 mg, more preferably between 1.0 to 30 mg.

β-Cryptoxanthin: For usual applications the daily dosage for humans (usually determined for a 70 kg person) for β-cryptoxanthin not exceed 20 mg, preferably not exceed 15 mg. In some embodiments of the invention the daily dosage for humans (70 kg person) for β-cryptoxanthin between 0.1 to 20 mg, more preferably between 0.5 to 15 mg.

Zeaxanthin: daily dosage for humans (70 kg person): 0.1 to 20 mg, preferred daily dosage for humans (70 kg person): 2 to 7 mg, more preferred daily dosage for humans (70 kg person): ca. 4 mg.

Biotin: daily dosage for humans (70 kg person): preferred daily dosage for humans (70 kg person): more preferred daily dosage for humans (70 kg person).

Vitamin E: For humans (70 kg person) the daily dosage preferably may vary for vitamin E between 15 mg and 2 g, more preferably between 15 and 500 mg.

Vitamin C: For humans (70 kg person) the daily dosage preferably may vary for vitamin C between 100 mg and 5 g, more preferably between 200 mg and 1.5 g.

Vitamin B12: daily dosage for humans (70 kg person): 0.5 to 10 μg, preferred daily dosage for humans (70 kg person): 1 to 5 μg, more preferred daily dosage for humans (70 kg person): 2.4 to 3 μg.

CoQ-10: daily dosage for humans (70 kg person): 1 to 100 mg, preferred daily dosage for humans (70 kg person): 5 to 60 mg.

β-Carotene: daily dosage for humans (70 kg person): 0.1 to 50 mg, preferred daily dosage for humans (70 kg person): 1 and 30 mg, more preferred daily dosage for humans (70 kg person): 2 to 7 mg.

Genistein: daily dosage for humans (70 kg person): 1 to 150 mg, preferred daily dosage for humans (70 kg person): 20 to 60 mg, more preferred daily dosage for humans (70 kg person): 20 to 40 mg.

The same amounts as listed above apply if combinations as e.g. the following are used:

(−)-epigallocatechin gallate, genistein and resveratrol;
(−)-epigallocatechin gallate, genistein and β-carotene;
(−)-epigallocatechin gallate, resveratrol and β-carotene;
(−)-epigallocatechin gallate, resveratrol and a n-3/n-6 polyunsaturated fatty acid (derivative);
(−)-epigallocatechin gallate, β-carotene and a n-3/n-6 polyunsaturated fatty acid (derivative);
(−)-epigallocatechin gallate, genistein and a n-3/n-6 polyunsaturated fatty acid (derivative);
genistein, resveratrol and β-carotene;
resveratrol, genistein and a n-3/n-6 polyunsaturated fatty acid (derivative);
β-carotene, genistein and a n-3/n-6 polyunsaturated fatty acid (derivative);
β-carotene, resveratrol and a n-3/n-6 polyunsaturated fatty acid (derivative);
genistein, resveratrol, β-carotene and a n-3/n-6 polyunsaturated fatty acid (derivative);
(−)-epigallocatechin gallate, resveratrol, β-carotene and a n-3/n-6 polyunsaturated fatty acid (derivative);
(−)-epigallocatechin gallate, genistein, β-carotene and a n-3/n-6 polyunsaturated fatty acid (derivative);
(−)-epigallocatechin gallate, genistein, resveratrol and a n-3/n-6 polyunsaturated fatty acid (derivative);
(−)-epigallocatechin gallate, genistein, resveratrol and β-carotene;
(−)-epigallocatechin gallate, genistein, resveratrol, β-carotene and a n-3/n-6 polyunsaturated fatty acid (derivative) (especially preferred).

In an embodiment of the present invention the compounds, combinations and compositions as cited above may be also incorporated in suitable carriers to administer the active ingredients topically. In a further embodiment of the present invention the oral administration is combined with the topical administration. In a further embodiment of the present invention the oral or the oral-topical administration is combined with a stimulation of the skin, especially of the problem areas, mechanically by messages/scrubbing or by ultra-sound.

In one body of the invention the composition improves the constitution of the connective tissue, i.e. the extracellular matrix and interstitium in the dermis, and resolves fibrosclerosis and restructures the fibrotic septae (EGCG, vitamin A, provitamin A carotenoids, vitamin C). It reverses and cures the glucosamino glycane alterations present in cellulite interstitial dermis from the hydrophilic to the less hydrophilic conformation. In another aspect of the invention, the composition reduces the permeability of dermal capillaries, increases the tone and reduces vasodilatation and thus increases the microvasculature flow and improves subcutaneous microcirculation (EGCG, PUFAs, genistein). The composition thus causes a reduction in interstitial pressure and in the interstitial edema present in cellulite. Overall, this leads to alleviation of hypoxia and an improved oxygen supply with reversal and prevention of the blood stasis in cellulite subcutaneous tissue. In another embodiment of the invention, the composition also inhibits the subcutaneous adipocyte proliferation (EGCG, PUFA, vitamin A, carotenoids) and differentiation, and further prevents lipogenesis and reduces hypertrophy of adipocytes and also supports lipolysis.

Thus one aspect of the invention is that the composition not only reduces the subcutaneous fat tissue and reduces the size and appearance of micro nodules and macro nodules associated with cellulite but also prevents their protrusion into the dermis.

One other aspect of the invention is that the subcutaneous latent inflammation caused by the edematous pressure is reduced and alleviated.

An important aspect of the invention is that by treatment with the composition, dimpling is reduced, the micro relief of the skin is smoothened, and its orange peel like and lumpy-bumpy appearance is reduced and/or resolved. Skin elasticity and tensile properties are increased, the skin gets softer and exhibits improved hydration and looses its mattress-like appearance.

Another aspect of the invention is the reduction of fat mass and the circumference at the hips and thighs [=upper legs].

Another embodiment of the invention includes also the prevention of the development the above mentioned conditions by the composition in a person having not yet visible signs of cellulite, or the prevention and/or slowing of the progression from moderate/mild cellulite with a lower grade to a higher grade of cellulite.

A further important aspect of the invention is that the person in need of treatment is relieved from significant psychological pressure and stress due to the unpleasant cosmetic appearance and the overall quality of life is significantly improved.

The invention is illustrated further by the following examples.

### EXAMPLES

### Example 1

### Compounds Affecting Adipocyte Differentiation

### Compositions for Preventing Mammalian Adipocyte Differentiation

In the present invention, the effect of agents was identified by using cellular assays that measure the effect on adipocyte differentiation. Adherent pre-adipocyte cells, C3H10T1/2, were used; they were cultured in complete DMEM/FCS, i.e, Dulbecco's modified essential medium (DMEM) supplemented with penicillin/streptomycin [50 U penicillin/ml, 50 μg streptomycin/ml] and 10% fetal calf serum (FCS) and passaged every 3-5 days. For experiments, C3H10 T1/2 cells were re-suspended in DMEM/FCS without phenol red and seeded at ˜x10⁵ cells/per well in collagen I pre-coated 24-well plates. After 2-3 days cells reached confluence. Culture medium was changed and cell differentiation was induced by adding induction medium consisting of rosiglitazone and insulin (at 50 μmol/L and 200 nmol/L, respectively). In some cases the induction medium consisted of 1 μmol/L dexamethasone and 500 mmol/L 3-isobutyl-1-methylxanthine. Control cells were cultured in DMEM/FCS containing only insulin. Where appropriate, substances were added to the differentiation medium at various concentrations at the time of induction, except for carotenoids which were added at the beginning of culture in order to preload the cells. The induction medium was changed every 2-3-days. Seven days after induction, cells were fixed in 60% isopropanol (40% water). After two washes in phosphate buffered saline (PBS), cells were stained with the neutral lipid stain Bodipy® 493/505 (0.5 μg/ml) and the nuclear stain Hoechst 33342 dye (2 μg/ml) for 30 minutes in the dark. After washing with PBS, the stained cells were stored at 4° C. in PBS. The extent of differentiation was quantified in a Cellomics Array™ Array Scan V^{Ti} (http://www.cellomics.com/) using the Spot Detector BioApplication that had been optimized for detecting lipid accumulation in differentiated adipocytes. Effects of treatments were quantified using the ‘SpotCount’ and ‘SpotCountPerObject’ parameter that adequately reflects the differentiation status of the adipocytes.

The tested compounds were purchased from Sigma (PUFAs, i.e. DHA docosahexaenoic acid; EPA eicosopentaenoic acid) or Cayman Chemicals (hydroxytyrosol), from DSM (hydroxytyrosol, resveratrol, genistein) or from Ehrenstorfer GmbH, Augsburg Germany (carotenoids like β-carotene, β-cryptoxanthin, lutein, lycopene, zeaxanthin); rose hip concentrate/powder was purchased from HybenVital (Denmark), The compounds to be tested were dissolved in dimethylsulfoxide (DMSO) or—in the case of carotenoids—in tetrahydrofuran (THF). Cells were preloaded with carotenoids, i.e. these were added to the culture medium when cells were seeded into test plates. Carotenoids were freshly prepared for every experimental series. Vehicle solution was added to reach a final concentration of 0.5% (v/v).

In some cases, cell viability was determined by measuring LDH concentrations in culture medium at the end of the treatment period. No significant differences in LDH release was observed in the different treatment groups, indicating absence of deleterious effects of these compounds on cell viability during the culture period.

### Effects on Adipocyte Differentiation

Compounds were tested in the micromolar range (in the case of defined substances) or in the mg/L range (in the case of plant extracts (like rosehip concentrate/powder). Inducers of differentiation triggered substantial accumulation of lipids (in form of droplets) in C3H10T1/2 cells (set at 100% in Table 1). We found in the present invention that many tested substances significantly reduced the extent of in vitro induced differentiation of pre-adipocytes to adipocytes since accumulation of cytoplasmic lipid droplets was diminished (Table 1). For instance, hydroxytyrosol, DHA, EPA, some carotenoids and rose hip concentrate/powder significantly reduced the amount of cytoplasmic lipids. Conversely, EGCG, resveratrol and genistein had only minor effects even at the highest concentrations tested. We further tested the effect of substances that were added to cultures concomitantly. The obtained values were then compared to the sum of the effect of individual substances: positive values indicate that two substances interact synergistically and thus surpass the individual effects. Surprisingly, the combination of distinct substances induced effects that exceeded those expected by the sum of the single compounds' effects (Table 2), thus reflecting a substantial synergistic effect. In particular, the resveratrol/rosehip concentrate combination reflected a strong synergistic effect (see % differences between expected and observed % of inhibition in Table 2). Also, resveratrol and EGCG or genistein reflected synergistic effect. This is even more surprising, since these compounds had no significant effect when tested alone. In addition, many other combinations exhibit synergistic effects on adipocyte differentiation. Very strong synergistic effects were notably observed with the combinations involving rose hip concentrate/powder/hydroxytyrosol, hydroxytyrosol/docosahexaeonic acid (DHA), β-carotene/genistein and β-carotene/rose hip concentrate/powder. Most importantly, the combination of β-carotene with four other substances also reflected synergistic interactions of five different compounds.

TABLE 1
Lipid accumulation in adipocytes. Cells were induced to in vitro
and cultured for 7 days with the indicated substances. The lipid
contents were quantified by Cellomics ™ HCS technology

| | Cellular lipids | |
|---|---|---|
| | (in % of | |
| | differentiated | |
| Reagents | cells) | p value¹⁾ |
| none | 0.0 | |
| Inducers of | 100.0 | |
| differentiation²⁾ | | |
| ⁶⁾+25 μM EGCG | 93.1 | 0.415 |
| +5 μM EGCG | 98.9 | 0.874 |
| +1 μM EGCG | 113.1 | 0.216 |
| +25 μM Genistein | 95.9 | 0.442 |
| +5 μM Genistein | 99.7 | 0.898 |
| +1 μM Genistein | 111.5 | 0.104 |
| +25 μM Resveratrol | 102.8 | 0.752 |
| +5 μM Resveratrol | 102.4 | 0.805 |
| +1 μM Resveratrol | 105.7 | 0.621 |
| +25 μM Hydroxytyrosol | 68.3 | 0.077 |
| +5 μM Hydroxytyrosol | 102.6 | 0.772 |
| +1 μM Hydroxytyrosol | 102.7 | 0.176 |
| +25 μM DHA³⁾ | 66.2 | 0.046 |
| +5 μM DHA⁴⁾ | 91.5 | 0.617 |
| +1 μM DHA | 95.9 | 0.779 |
| +25 μM EPA | 74.1 | 0.005 |
| +5 μM EPA | 77.0 | 0.018 |
| +1 μM EPA | 86.6 | 0.044 |
| +250 μg/ml RH⁵⁾ | 84.3 | 0.080 |
| +50 μg/ml RH | 102.2 | 0.683 |
| +10 μg/ml RH | 108.1 | 0.323 |
| +2.0 μM lutein | 72.1 | 0.047 |
| +1.0 μM lutein | 85.6 | 0.081 |
| +0.5 μM lutein | 94.3 | 0.615 |
| +2.0 μM β- | 16.2 | 0.002 |
| cryptoxanthin | | |
| +1.0 μM β- | 34.9 | 0.015 |
| cryptoxanthin | | |
| +0.5 μM β- | 47.5 | 0.030 |
| cryptoxanthin | | |
| +2.0 μM zeaxanthin | 43.7 | 0.011 |
| +1.0 μM zeaxanthin | 39.5 | 0.012 |
| +0.5 μM zeaxanthin | 56.8 | 0.285 |
| +2.0 μM lycopene | 24.6 | 0.000 |
| +1.0 μM lycopene | 45.1 | 0.017 |
| +0.5 μM lycopene | 44.5 | 0.058 |
| +2.0 μM beta-carotene | 69.4 | 0.145 |
| +1.0 μM beta-carotene | 66.7 | 0.173 |
| +0.5 μM beta-carotene | 93.0 | 0.611 |

¹⁾statistical significance of difference calculated on the triplicate values of the raw data (spot per object). Bold numbers: p < 0.1
²⁾see text for inducer of differentiation
³⁾DHA: docosahexaenoic acid
⁴⁾EPA: eicosopentaenoic acid
⁵⁾RH: rosehip concentrate/powder
⁶⁾Inducers of differentiation + compound

TABLE 2
Synergistic effects of substances on adipocyte differentiation.
Cells were induced to differentiate in the presence of the indicated substances and the percentage of
inhibition determined (see Table 1); the synergistic interactions were calculated as detailed in the text.
Positive values indicated below reflect synergistic effects of compounds at the indicated concentrations.

| | | | | | | | | | | | | | 250 | 50 | 10 |
|---|---|---|---|---|---|---|---|---|---|---|---|---|---|---|---|
| | 25 uM | 5 uM | 1 uM | 25 uM | 5 uM | 1 uM | 25 uM | 5 uM | 1 uM | 25 uM | 5 uM | 1 uM | ug/ml | ug/ml | ug/ml |
| | Gen | Gen | Gen | Resv | Resv | Resv | OH-Ty | OH-Ty | OH-Ty | DHA | DHA | DHA | RH | RH | RH |
| 25 uM EGCG | | | | 20.7 | | | | | | | | | 24.8 | | |
| 5 uM EGCG | | | | | | | | 7.6 | | | | | | 8.7 | |
| 1 uM EGCG | | | 28.3 | | | 2.5 | | | 16.8 | | | | | | 18.8 |
| 25 uM Genistein | | | | 22.6 | | | | | | | | | | | |
| 5 uM Genistein | | | | | | | | | | | | | | | |
| 1 uM Genistein | | | | | | 16.5 | | | 26.5 | | | | | | |
| 25 uM Resveratrol | | | | | | | | | | 23.4 | | | 72.4 | | |
| 5 uM Resveratrol | | | | | | | | 11.1 | | | 3.3 | | | 28.9 | |
| 1 uM Resveratrol | | | | | | | | | 6.8 | | | 14.8 | | | 16.4 |
| 25 uM EPA | | | | | | | 31.8 | | | | | | | | |
| 5 uM EPA | | | | | | | | 9.0 | | | 42.3 | | | | |
| 1 uM EPA | | | | | | | | | 25.2 | | | 39.1 | | | 7.1 |
| 25 uM Hydroxytyrosol | | | | | | | | | | 51.9 | | | | | |
| 5 uM Hydroxytyrosol | | | | | | | | | | | 71.2 | | | 7.3 | |
| 1 uM Hydroxytyrosol | | | | | | | | | | | | 87.4 | | | 24.4 |
| 2 uM b-carotene | 11.6 | | | 23.1 | | | | | | | | | 25.5 | | |
| 1 uM b-carotene | | 32.9 | | | | | | | | | 35.5 | | | 6.5 | |
| 0.5 uM b-carotene | | | 61.3 | | | | | | 17.2 | | | 53.8 | | | 31.0 |
| | Combined with EGCG (1 uM) + Resveratrol (1 uM) + Genistein (1 uM) + RH (10 mg/L) | | | | | | | | | | | | | | |
| 0.5 uM b-carotene | | | 6.9 | | | | | | | | | | | | |

Abbreviations used in Table 2:
“Gen” = genistein,
“OH-Ty” = Hydroxytyrosol;
“Resv” = Resveratrol;
“uM” = micromolar (μM).

### Example 2

### Preparation and Administration of a Dietary Supplement (Capsule 1) According to the Present Invention

Lecithin is dissolved in the ROPUFA ‘30’ N-3 Food oil (triglyceride, containing min. 30% n-3 PUFAs, stabilized with mixed tocopherol, ascorbyl palmitate and rosemary extracts; commercially available from DSM Nutritional Products AG, Kaiseraugst, Switzerland) and the beta-Carotene 30% FS (commercially available from DSM Nutritional Products AG, Kaiseraugst, Switzerland) dispersion is added. Teavigo, genistein and resveratrol are mixed in a tumbler mixer for 5 minutes. This dry powder mix is dispersed in the oily mixture of ROPUFA, carotene and lecithin and then encapsulated in soft gel capsules.

A female may take 4 capsules a day at least for 3-6 months. The intake may result in an alleviation of already existing cellulite, in the prevention of the development of mild cellulite or in the prevention of progression of mild to severe cellulite, in the reduction of the fat mass and the circumference at the hips and thighs, in the smoothening of the micro relief of the skin, in the maintenance or increase of the tensile properties of the skin, in the maintenance of a smooth and firm skin and/or in the beautification of the silhouette/bodyshape.

The amounts are given in the following table 3.

| | | | | amount |
|---|---|---|---|---|
| | daily | label | conc. of active | product |
| | dossage of | (mg)/ | in product | form mg/ |
| Capsule recipe 1 | active (mg) | capsule | form (%) | capsule |
| Teavigo (EGCG) | 150 | 37.5 | 90 | 41.7 |
| Genistein | 30 | 7.5 | 100 | 7.5 |
| Resveratrol | 25 | 6.25 | 100 | 6.3 |
| beta-Carotene | 7 | 1.75 | 30 | 5.8 |
| 30% FS | | | | |
| ROPUFA ‘30’ n-3 | 1000 | 250 | 25 | 1000.0 |
| Food oil | | | | |
| Lecithin | | | | 5.4 |
| Sum | | | | 1066.7 |

### Example 3

### Preparation and Administration of a Dietary Supplement (Capsule 2) According to the Present Invention

Lecithin is dissolved in the ROPUFA ‘75’ N-3 EE (ethyl ester containing min. 72% n-3 PUFAs, stabilized with mixed tocopherol, ascorbyl palmitate, citric acid and rosemary extracts; commercially available from DSM Nutritional Products AG, Kaiseraugst, Switzerland) and the beta-Carotene 30% FS (commercially available from DSM Nutritional Products AG, Kaiseraugst, Switzerland) dispersion is added. Teavigo, genistein and resveratrol are mixed in a tumbler mixer for 5 minutes. This dry powder mix is dispersed in the oily mixture of ROPUFA, carotene and lecithin and then encapsulated in soft gel capsules.

A female may take 2 capsules a day at least for 3-6 months. The intake may result in an alleviation of already existing cellulite, in the prevention of the development of mild cellulite or in the prevention of progression of mild to severe cellulite, in the reduction of the fat mass and the circumference at the hips and thighs, in the smoothening of the micro relief of the skin, in the maintenance or increase of the tensile properties of the skin, in the maintenance of a smooth and firm skin and/or in the beautification of the silhouette/bodyshape.

The amounts are given in the following table 4.

| Capsule recipe 2 | | | | |
|---|---|---|---|---|
| Teavigo | 150 | 75 | 90 | 83.3 |
| Genistein | 30 | 15 | 100 | 15.0 |
| Resveratrol | 25 | 12.5 | 100 | 12.5 |
| beta-Carotene 30% | 7 | 3.5 | 30 | 11.7 |
| FS | | | | |
| ROPUFA ‘75’ n-3 EE | 1000 | 500 | 72 | 694.4 |
| Lecithin | | | | 8.1 |
| Sum | | | | 825.0 |

### Example 4

### Preparation and Administration of a Fortified Food (Fortified Yoghurt) According to the Present Invention

Fermented Yoghurt drink (probiotic) 1.5% fat

| Ingredients | Weight (g) |
|---|---|
| Whole milk 1.7% | 875.42 |
| Skim milk 1.0% | 10.00 |
| Nestle LC1 Yoghurt | 30.00 |
| Sugar | 70.00 |
| Stabiliser | 3.00 |
| Tangerine flavour | 0.08 |
| Teavigo | 0.93 |
| Genistein TG | 0.17 |
| Resveratrol | 0.14 |
| β-Carotene 7% CWS | 0.24 |
| ROPUFA ‘30’ n-3 Food oil | 10.00 |
| Total fermented drink | 1000.00 |

| | | Serving | |
|---|---|---|---|
| New health ingredient | Product form [%] | [g] | [mg/serving] |
| Teavigo ® | min. 90 | 60 g | 50 |
| Genistein TG | 99% | | 10 |
| Resveratrol | 100% | | 8.3 |
| β-Carotene 7% CWS | min. 7% | | 1.0 |
| ROPUFA ‘30’ n-3 Food oil | min. 25% LCP | | 150.0 |

Subjects should eat 3 servings a day at least for 3-6 months. The intake may result in the reduction of the fat mass and the circumference at the hips and thighs, in the smoothening of the micro relief of the skin, in the maintenance or increase of the tensile properties of the skin, in the maintenance of a smooth and firm skin and/or in the beautification of the silhouette/bodyshape.

### Preparation

Warm milk to 40° C.

Add dry ingredients and colour and heat to 65° C.

Homogenise at 65° C. P1: 150 bar/P2: 50 bar

Pasteurise at 90° C., 20 min

Cool down to 45° C.

Add starter culture and stir

Ferment at 45° C. until the pH has reached 4.3

Cool down, add flavour and stir

Homogenise at 25° C., 30 bar.

Cool down to 10° C.

Package and store at cool temperatures.

### Example 5

### Preparation and Administration of a Fortified Food (Fortified Cereal Bar) According to the Present Invention

| Ingredients | Quantity [g] |
|---|---|
| Sugar | 118.0 |
| Water | 54.0 |
| Salt | 1.5 |
| Glucose syrup DE38, 43°Be | 130.0 |
| Invert sugar syrup (74-76%) | 95.0 |
| Sorbitol syrup | 35.0 |
| Palmkernel fat | 60.0 |
| Biscofin N | 40.0 |
| Lecithin | 1.5 |
| Monomuls 90-35-5 (emulsifer) | 2.5 |
| Apple dried and cut | 63.0 |
| Raisins | 27.0 |
| Cornflakes | 100.0 |
| Rice crispies | 140.0 |
| Mini Crispini, Wheat | 90.0 |
| Hazelnut, roasted | 54.0 |
| Skim milk powder | 45.0 |
| Apple flavour 74863-33 | 2.0 |
| Citric acid* | 5.0 |
| Teavigo ® | 1.85 |
| Genistein TG | 0.34 |
| Resveratrol | 0.28 |
| β-Carotene 10% B | 0.77 |
| ROPUFA ‘10’ n-3 Food Powder S/SD | 20.0 |
| Yield | 1000.0 |

*used to support the apple flavour

| | | Serving | |
|---|---|---|---|
| New health ingredient | Product form [%] | [g] | [mg/serving] |
| Teavigo ® | min. 90 | 30 g | 50 |
| Genistein TG | 99% | | 10 |
| Resveratrol | 100% | | 8.3 |
| β-Carotene 10% B | min. 10% | | 2.3 |
| ROPUFA ‘10’ n-3 Food | min. 7% LCP | | 42.0 |
| Powder S/SD | | | |

Subjects should eat 3 servings a day at least for 3-6 months. The intake may result in the reduction of the fat mass and the circumference at the hips and thighs, in the smoothening of the micro relief of the skin, in the maintenance or increase of the tensile properties of the skin, in the maintenance of a smooth and firm skin and/or in the beautification of the silhouette/bodyshape.

### Preparation

Premix Teavigo ®, Genistein TG, Resveratrol, β-Carotene 10%
B and ROPUFA ‘10’ n-3 Food Powder S/SD with skim milk powder
and place in a Kenwood type mixer
Add cornflakes, ricecrispies and gently mix with 2.1. Then add the
more humid ingredients as dried apples and raisins. All ingredients are
gently mixed in order to ensure a good distribution of the dry ingredients
The following ingredients are weight into a separate bowl each
Sugar, water, salt
Glucose-inverte and sorbitol syrup
Biscofin N, Palmkernel fat, Lecithin and Emulsifier
Mixture of sugar, water and salt is heated to 110° C.
Mixture of the different syrups is heated to 113° C. and cooled in a
cold water bath in order to stop the cooking process
Solution 2.3 and 2.4 are combined
Mixture of Biscofin N, palm kernel fat, lecithin and emulsifier are molten
in a water bath at 75° C.
Mixture (2.6) of fats is added to the combined sugar solution (2.5). The
later should be still hot
Flavour and citric acid is added to the liquid mass (2.7)
The liquid mass is added to the dry ingredients (2.2) in the Kenwood
mixer and mixed well with the dry ingredients
The mass is put on a marmor plate and rolled to the desired thickness.
Then the mass is cooled down at room temperature
Cut into pieces of e.g. one serving size and pack into e.g. aluminum bags

Capsules, fortified yoghurts and fortified cereal bars containing (−)-epigallocatechin gallate, genistein, resveratrol, β-carotene and a rose hip extract/concentrate may be prepared in an analogous way to examples 2-5.

1.-56. (canceled)

57. Cosmetic or medical method for smoothening the micro relief of the skin, for the maintenance of a smooth and firm skin and/or for the beautification of the silhouette/bodyshape by administering to a subject in need thereof an effective amount of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof.

58. Cosmetic or medical method for maintaining or increasing the tensile properties of the skin by administering to a subject in need thereof an effective amount of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof.

59. Cosmetic or medical method for the treatment of cellulite, for the prevention of the development of mild cellulite, for the prevention of the progression of mild cellulite to severe cellulite by administering to a subject in need thereof an effective amount of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof.

60. Cosmetic or medical method for reducing the fat mass and the circumference at the hips and thighs by administering to a subject in need thereof an effective amount of at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β cryptoxanthin, zeaxanthin and derivatives thereof.

61. Cosmetic or medical method for the treatment of cellulite, for the prevention of the development of mild cellulite, for the prevention of the progression of mild cellulite to severe cellulite, for smoothening the micro relief of the skin, for maintaining or increasing the tensile properties of the skin, for reducing the fat mass and the circumference at the hips and thighs, for the maintenance of a smooth and firm skin and/or the beautification of the silhouette/bodyshape by administering to a subject in need thereof an effective amount of a composition comprising (−)-epigallocatechin gallate and resveratrol.

62. Cosmetic or medical method for the treatment of cellulite, for the prevention of the development of mild cellulite, for the prevention of the progression of mild cellulite to severe cellulite, for smoothening the micro relief of the skin, for maintaining or increasing the tensile properties of the skin, for reducing the fat mass and the circumference at the hips and thighs, for the maintenance of a smooth and firm skin and/or the beautification of the silhouette/bodyshape by administering to a subject in need thereof an effective amount of a composition comprising resveratrol and genistein.

63. Cosmetic or medical method for the treatment of cellulite, for the prevention of the development of mild cellulite, for the prevention of the progression of mild cellulite to severe cellulite, for smoothening the micro relief of the skin, for maintaining or increasing the tensile properties of the skin, for reducing the fat mass and the circumference at the hips and thighs, for the maintenance of a smooth and firm skin and/or the beautification of the silhouette/bodyshape by administering to a subject in need thereof an effective amount of a composition comprising a n-3/n-6 polyunsaturated fatty acid (derivative) and lycopene.

64. Cosmetic or medical method for the treatment of cellulite, for the prevention of the development of mild cellulite, for the prevention of the progression of mild cellulite to severe cellulite, for smoothening the micro relief of the skin, for maintaining or increasing the tensile properties of the skin, for reducing the fat mass and the circumference at the hips and thighs, for the maintenance of a smooth and firm skin and/or the beautification of the silhouette/bodyshape by administering to a subject in need thereof an effective amount of a composition comprising a n-3/n-6 polyunsaturated fatty acid (derivative) and lutein.

65. Cosmetic or medical method for the treatment of cellulite, for the prevention of the development of mild cellulite, for the prevention of the progression of mild cellulite to severe cellulite, for smoothening the micro relief of the skin, for maintaining or increasing the tensile properties of the skin, for reducing the fat mass and the circumference at the hips and thighs, for the maintenance of a smooth and firm skin and/or the beautification of the silhouette/bodyshape by administering to a subject in need thereof an effective amount of a composition comprising (−)-epigallocatechin gallate, resveratrol and a n-3/n-6 polyunsaturated fatty acid (derivative).

66. Cosmetic or medical method for the treatment of cellulite, for the prevention of the development of mild cellulite, for the prevention of the progression of mild cellulite to severe cellulite, for smoothening the micro relief of the skin, for maintaining or increasing the tensile properties of the skin, for reducing the fat mass and the circumference at the hips and thighs, for the maintenance of a smooth and firm skin and/or the beautification of the silhouette/bodyshape by administering to a subject in need thereof an effective amount of a composition comprising—epigallocatechin gallate, resveratrol, β-carotene and a n-3/n-6 polyunsaturated fatty acid (derivative).

67. Cosmetic or medical method for the treatment of cellulite, for the prevention of the development of mild cellulite, for the prevention of the progression of mild cellulite to severe cellulite, for smoothening the micro relief of the skin, for maintaining or increasing the tensile properties of the skin, for reducing the fat mass and the circumference at the hips and thighs, for the maintenance of a smooth and firm skin and/or the beautification of the silhouette/bodyshape by administering to a subject in need thereof an effective amount of a composition comprising hydroxytyrosol and docosahexaenoic acid.

68. Cosmetic or medical method for the treatment of cellulite, for the prevention of the development of mild cellulite, for the prevention of the progression of mild cellulite to severe cellulite, for smoothening the micro relief of the skin, for maintaining or increasing the tensile properties of the skin, for reducing the fat mass and the circumference at the hips and thighs, for the maintenance of a smooth and firm skin and/or the beautification of the silhouette/bodyshape by administering to a subject in need thereof an effective amount of a composition comprising β-carotene and genistein.

69. The method according to claim 57, wherein the administering is oral.

70. Cosmetic or medical method for the treatment of cellulite, for the prevention of the development of mild cellulite, for the prevention of the progression of mild cellulite to severe cellulite, for smoothening the micro relief of the skin, for maintaining or increasing the tensile properties of the skin, for reducing the fat mass and the circumference at the hips and thighs, for the maintenance of a smooth and firm skin and/or the beautification of the silhouette/bodyshape by administering to a subject in need thereof an effective amount of a composition comprising (−)-epigallocatechin gallate, genistein, resveratrol, β-carotene and a n-3/n-6 polyunsaturated fatty acid (derivative).

71. A dietary, cosmetic or dermatological composition for the treatment of cellulite in female humans, for the prevention of the development of mild cellulite in female humans, for the prevention of the progression of mild cellulite to severe cellulite in female humans, for smoothening the micro relief of the skin of female humans, for maintaining or increasing the tensile properties of the skin of female humans, for reducing the fat mass and the circumference at the hips and thighs of female humans, for the maintenance of a smooth and firm skin of female humans and/or for the beautification of the silhouette/bodyshape of female humans, said composition containing at least one compound selected from the group consisting of (−)-epigallocatechin gallate, resveratrol, n-3 polyunsaturated fatty acids, rose hip extract/concentrate, an olive polyphenol such as hydroxytyrosol; lycopene, lutein, β-cryptoxanthin, zeaxanthin and derivatives thereof.

72. The dietary composition according to claim 57 being in form of food such as dairy products (yoghurts), in form of fortified food such as cereal bars and bakery items such as cakes and cookies, in form of dietary supplements such as tablets, pills, granules, dragées, capsules, instant drinks and effervescent formulations, in form of nonalcoholic drinks such as soft drinks, sport drinks, fruit juices, lemonades, teas, near water drinks, and milk based drinks, in form of liquid food such as soups and dairy products (muesli drinks) or in form of beauty foods and supplements.
